(19) United States
(12) Patent Application Publication
(10) Pub. No.: US 2009/0142749 A1
(43) Pub. Date: Jun. 4, 2009

(54) Assessment of disease risk by quantitative determination of epimutation in normal tissues

(75) Inventors: Robyn Lynne Ward, Woollahra (AU); David I.K. Martin, Berkeley, CA (US); Catherine Mary Suter, Enmore (AU)

Correspondence Address: FOLEY AND LARDNER LLP;SUITE 500, 3000 K STREET NW, WASHINGTON, DC, 20007, US

(73) Assignees: ST. VINCENT'S HOSPITAL (SYDNEY) LIMITED; VICTOR CHANG CARDIAC RESEARCH INSTITUTE

(21) Appl. No.: 10/576,575

(22) PCT Filed: Oct. 20, 2004
(86) PCT No.: PCT/AU04/01434

§ 371 Date: Jul. 14, 2008

Related U.S. Application Data
(60) Provisional application No. 60/513,097, filed on Oct. 20, 2003.

(30) Foreign Application Priority Data
Oct. 20, 2003 (AU) 2003905747

(51) Int. Cl. C12Q 1/68

(52) U.S. Cl. 435/6

(57) ABSTRACT

An assay for assessing the risk of disease in an individual, wherein said assay comprises the steps of isolating a population of cells from normal tissue of said individual, and quantitatively determining the frequency of epimutation of a particular gene in said population of cells, wherein the epimutation of said gene is associated with said disease and said gene is other than one that is subject to normal parent of origin-specific expression. Preferably, the epimutation is DNA methylation of a tumour suppressor gene such as hMLH1, hMSH2, APC 1A, APC 1B and p16.

### FIELD OF THE INVENTION

The present invention relates to an assay for assessing the risk of disease (e.g. cancer) in an individual. In particular, the present invention relates to an assay for assessing the risk of disease comprising quantitatively determining the frequency of an epimutation in a particular gene in a population of cells from normal tissue of an individual, wherein epimutation of the gene is associated with one or more diseases.

### BACKGROUND OF THE INVENTION

### Epigenetic Modifications and Gene Expression

Epigenetic modifications are molecular events that result in alterations in gene function that are mediated by factors other than a change in DNA sequence. Epigenetic effects on gene function commonly result in transcriptional silencing of the gene that may be maintained through mitosis, producing clonal patterns of transcriptional silence. Silencing may occur with a probability that is somewhere between 0 and 1, producing, in a single multicellular organism, a mosaic pattern of gene expression (or silence). This mosaic expression occurs despite all cells having the same genetic makeup. In some cases, epigenetic modifications are maintained in the germ-line, producing heritable effects (“epigenetic inheritance”). The molecular basis of epigenetic effects is much more complex than the simple 4-base code in DNA, and for this reason, epigenetic inheritance occurs in patterns that are much different from the simple patterns of Mendelian inheritance.

One of the best known epigenetic modifications is cytosine methylation (“DNA methylation”), which is indispensable for normal human development and is involved in the normal physiological processes of parental imprinting, suppression of transposable elements, and X-inactivation in females (reviewed in Jones and Takai 2001, and Bird 2002). In all mammals, cytosine methylation occurs essentially within the dinucleotide CpG. In the human genome, the majority of cytosine residues within CpG dinucleotides are methylated, but small proportions are maintained as unmethylated in certain CpG-rich regions called “CpG islands” (Antequera and Bird 1993). CpG islands are frequently associated with the regulatory regions of cellular genes, and a large proportion of human genes include a CpG island at their 5′ end.

Histone and chromatin structure changes are other epigenetic modifications which affect gene expression. Indeed, both of these kinds of epigenetic modifications have been found to have a great impact on gene expression that is linked, although not exclusive to, DNA methylation within CpG islands (Jenuwein and Allis 2001). For example, transcriptionally active genes are generally associated with the acetylation of the fourth lysine (K⁴) of the histone subunit 3 (H3K⁴), whereas silent and methylated genes are correlated with de-acetylated H3K⁴, methylation of H3K⁹, and recruitment of the HP1 chromodomain (Kouzarides 2002). In fact, recent evidence indicates that DNA methylation occurs in response to a change in chromatin structure that is largely dictated by modifications to these key histone subunits (Tamaru and Selker 2001), and indicates that the role of DNA methylation is a consolidation of the already silent state. Therefore, DNA methylation can be regarded as a “signature” of a stably silenced genetic locus.

DNA methylation is not, however, an absolute requirement or “signature” for gene silencing since many non-human species which are devoid of CpG methylation still exhibit epigenetic silencing phenomena. Therefore, in the human genome, there are presumably many genes devoid of CpG island promoters that will still be susceptible to epigenetic modification mediated by changes in histones and chromatin structure, rather than DNA methylation. The ease and low cost with which DNA methylation can be assayed, however, makes it an attractive target to search for epigenetic modifications in humans.

### Methods to Detect Epigenetic Changes

As described above, the simplest way to determine an epigenetic change is to test for CpG methylation. Traditionally, CpG methylation analysis has been carried out by Southern hybridisation, which assesses methylation-sensitive restriction enzyme sites within CpG islands of known genes, however, recently, more sophisticated methods for determining CpG methylation such as COBRA (combined bisulfite restriction analysis; Xiong and Laird 1997), bisulfite allelic sequencing (Frommer et al 1992), and MSP (methylation-specific PCR), have become available and allowed a more detailed analysis of CpG methylation across a CpG island of interest.

In particular, bisulfite modification of DNA now allows the discrimination of methylated CpG from unmethylated CpG, since the bisulfite treatment converts unmethylated cytosine to uracil through deamination whereas 5-methylcytosine is protected from deamination and thereby remains unchanged. Following treatment with bisulfite, the method requires that the bisulfite-modified sequence be amplified by PCR with strand-specific primers to yield a product in which uracil residues are amplified as thymine, and only 5-methylcytosine residues are amplified as cytosine. The PCR products can then be readily digested with restriction enzymes to distinguish methylated from unmethylated alleles (COBRA), or cloned and sequenced to provide methylation maps of individual DNA strands.

MSP, another widely used methylation assay method, can assess the methylation status of CpG sites within a CpG island, independent of the use of methylation-sensitive restriction enzymes. In this method, bisulfite modification is followed by amplification with primers specific for methylated DNA only, and results in the amplification of any hypermethylated alleles within a given sequence (U.S. Pat. No. 5,786,146).

Further, methods for detecting epigenetic modifications are not limited to analysis of CpG methylation. That is, epigenetic modifications can also be detected by various methods which assay specific proteins bound to transcriptionally active or silent regions of DNA, or protein modifications associated with active or silent states (e.g. detection of specific modifications of histones, and the detection of other proteins such as homologues of HP1). At present, these protein modifications are generally assayed by immunoprecipitation with antibodies and subsequent analysis for DNA sequences present in the precipitated material.

### Genetic Basis of Disease

Efforts to predict predisposition for a disease, such as cancer, have been to date based largely on age, personal or family history, or occasionally by the inheritance of genetic abnormalities (i.e. germ-line mutations). For example, mutations in the BRCA genes are present in around half of all individuals with a strong family history of breast cancers. However, this accounts for less than 1% of breast cancers overall, and in the great majority of remaining cases there is no familial pattern consistent with a defect in a single gene. A similar situation exists with a large number of other diseases. Indeed, Table 1 provides a list of over sixty diseases which have been linked to certain genes, but where only a relatively small proportion of cases can be explained by a single predisposing genetic change.

A common explanation for diseases that have some familial pattern indicating inheritance, but no evidence for a single predisposing genetic change, is that disease results from the interaction of multiple genes. In line with this, a trait (which can manifest as disease) may be produced by the combined action of several genes, but only certain alleles of those genes will contribute to the trait. Such traits have been termed “quantitative traits”, “complex traits” and “polygenic traits”, and diseases that may result from such a mechanism are typically known as “multifactorial” or “polygenic” (for reviews, see Risch 2000, and Botstein and Risch 2003).

Genetic studies can not therefore, predict predisposition to disease in most cases. However, the fact that only a few patients in a family showing a strong history of a disease such as breast cancer are likely to show a single predisposing genetic change, suggests that many individuals have an innate predisposition to cancer, the basis of which is currently unknown.

### Epimutations

A gene may be inactivated by epigenetic modification. The term “epimutation” was first defined by Holliday (Holliday 1987) as a “mitotically heritable change in the methylation of a gene”, however the term has since been extended to refer also to the other types of epigenetic modifications. As used herein, the term “epimutation” refers to any abnormal silencing of gene expression, in the absence of DNA sequence alteration. This definition specifically excludes abnormal silencing of a gene that is normally subject to parental imprinting (also termed “genomic imprinting” or simply “imprinting”). Parental imprinting is a normal process that involves changes in the transcription state of one allele of a gene determined by the parental origin of the allele (i.e. a change in the transcription state of one allele of a gene that is normally subject to parent of origin-specific expression). This process sometimes is aberrant, resulting in the loss of monoallelic expression and thus expression that is either biallelic or completely absent.

### PRIOR ART

To date, the only clear case of a germ-line epimutation comes from a naturally occurring variant of the flowering plant, toadflax (Linaria vulgaris) (Cubas et al 1999). In this example, biallelic methylation and transcriptional silencing of a gene controlling symmetry, Lcyc, was found to be the cause of an alteration in flower phenotype. The phenotype was found to be somewhat unstable with a tendency to revert (i.e. some flowers on mutant plants exhibited a wild type appearance and this was associated with a loss of Lcyc methylation). Plants exhibiting the mutant phenotype were able to transmit the epimutation to their progeny through the germ-line. Germ-line and soma are not well separated in plants however, and this may explain the relatively stable existence of this epimutation over at least 250 years.

Epimutations are common in tumour cells. There is now a large body of literature documenting epimutations in many types of tumour, and their inverse relationship to activity of the affected gene (for reviews, see Jones and Laird 1999, Wolffe and Matzke 1999, and Baylin and Herman 2000). In some cases, the epigenetic silencing of tumour suppressor genes gives rise to distinct tumour phenotypes. For example, in sporadic colorectal cancer, around 15% of tumours exhibit microsatellite instability (MSI). MSI is a hallmark of defective mismatch repair, but only a tiny fraction of these cancers will be explained by a genetic alteration in a mismatch repair gene. It is now known that bi-allelic methylation of the hMLH1 gene promoter is responsible for MSI tumours in the majority of cases (Herman et al 1998, and Wheeler et al 1999). MSI colorectal cancers also exhibit loss of imprinting (LOI) at IGF2 (Cui et al 1998), which may also have an epigenetic basis (Cui et al 2002). It has also been demonstrated that LOI could be detected not only in MSI tumours, but also in the normal tissues of such patients, including their peripheral blood (Cui et al 2003). The finding of LOI in peripheral blood in a small number of normal controls, and a large percentage of colorectal cancer patients has led to the hypothesis that LOI in peripheral blood is an indicator of colorectal cancer risk (U.S. Pat. No. 6,235,474).

Germ-line epimutations have not yet been described in humans, although a related phenomenon can be observed in a particular strain of inbred mice, the agouti viable yellow (A^{vy}). These mice carry the A^{vy} allele, in which an intracisternal A particle (IAP) retrotransposon is inserted at the 5′ end of the agouti (A) gene (Duhl et al 1994). When the IAP is epigenetically active, agouti transcription is initiated from a cryptic promoter within the 5′ LTR of the IAP. The tight tissue-specific expression of agouti is abrogated by the IAP, whose LTR is active in many or all tissues and, as a result, agouti may be expressed pancellularly in A^{vy} mice. It has been found that the CpG methylation of this IAP is inversely correlated with ectopic agouti expression, and this epigenetic modification appears to gives rise to a variation in phenotype in A^{vy} mice which includes not only yellow coat colour, but also obesity, Type II diabetes, and tumour susceptibility. Significantly, this phenotype is mosaic in many individuals, indicating that the IAP is active in some cells, and silent in others, in a clonal pattern. Further, recent data indicates that there is incomplete erasure of the A^{vy} IAP methylation between generations resulting in partial maternal inheritance of the epigenotype (Morgan et al 1999). This suggests that germ-line transmission of an epigenetic modification is possible in mammals.

### Need for Methods for Assessing Disease Risk

New and improved methods for assessing disease risk in individuals (i.e. predicting predisposition for a disease) are desirable. That is, knowledge of disease risk may allow for the adoption of preventative therapies and avoidance of disease risk factors, and may further assist in the identification of preferred therapies upon commencement of the disease or symptoms. Preferably, methods for assessing risk are relatively simple and either non-invasive or cause only minimal discomfort to individuals.

The present applicants have detected epimutation (i.e. CpG methylation) in the promoter of the tumour suppressor gene hMLH1, in normal tissues (e.g. peripheral blood) of cancer patients with tumours showing a loss of the hMLH1 protein, and have found, surprisingly, that the frequency of the detected epimutation in the cells of such normal tissues is predictive of the level of cancer risk.

### SUMMARY OF THE INVENTION

Thus, the present invention provides an assay for assessing the risk of disease in an individual, wherein said assay comprises the steps of;

(i) isolating a population of cells from normal tissue of said individual, and
(ii) quantitatively determining the frequency of epimutation of a particular gene in said population of cells, wherein epimutation of said gene is associated with said disease and said gene is other than one that is subject to normal parent of origin-specific expression.

### BRIEF DESCRIPTION OF THE FIGURES

FIG. 1 shows hMLH1 COBRA methylation analysis in peripheral blood in Example 1. (A) This photograph shows an example of the COBRA screening assay. In particular, results are shown from the C region COBRA in peripheral blood DNA from 44 cancer patients. In this subset, one patient showed methylation of hMLH1 in the C region demonstrated by digestion of the PCR product (upper band) to yield two smaller fragments, which appear as one band (arrow). (B) These photographs show the A, B and C region COBRA results for the peripheral blood of individuals VT and TT. The location of each region relative to the transcription start site is shown on the left. For each region, digestion of the PCR product (upper band) to yield smaller fragments is indicative of methylation within that region. +, RKO cell line; −, healthy control blood DNA.

FIG. 2 shows immunohistochemical analysis of hMLH1 expression in representative cancers from the two individuals in Example 1. Carcinomas in the upper panel are from VT (left=breast, middle=endometrium, right=colon) and those in the lower panel are from TT (left=colon, middle=ampulla of Vater. Right=duodenal). All cancers showed complete loss of hMLH1 expression. For all tumours, the inset shows positive staining of the same tumour for hMSH2. Immunoperoxidase with haematoxylin counterstain; Bar (lower left) represents 100 μm.

FIG. 3 shows bisulfite sequencing analysis of VT and TT somatic tissues in Example 1. (A) Schematic representation of the hMLH1 locus showing the locations of the A, B, and C regions in relation to the region sequenced (dotted lines). (B) Sequence of hMLH1 within the dotted region defined in (A). The primers used to amplify this region are underlined. CpG doublets within this domain are highlighted in bold and numbered 1 through 17. The single nucleotide polymorphism is also highlighted with G and A shown in larger text. (C) This figure shows the results of bisulfite allelic sequencing in the various somatic tissues of TT and VT. Black and white squares represent individual CpGs and are numbered according to their location in the sequence shown in (B). Grey or white circles represent the A or G genotype, respectively. Each horizontal row of squares represents the results from individual alleles. In both patient TT and VT, the hypermethylated alleles are always of the G genotype, whereas the A alleles show patchy methylation only and are never hypermethylated. Mosaicism was evident in the hair follicles of TT, and in all tissues from VT, as evidenced by the hypomethylation of the occasional G allele.

FIG. 4 shows the results of bisulfite sequencing analysis of TT sperm in Example 1. (A) This photograph shows the results of the hybrid MSP-COBRA PCR used to amplify methylated alleles from the purified sperm from patient TT. Note the weak amplification of sperm compared to the positive control cell line (+). No amplification was seen in the negative control (peripheral blood from a healthy donor). (B) This figure shows the results of bisulfite allelic sequencing of the fragment from sperm shown in (A). Black and white squares represent individual CpGs and are numbered according to their location in the sequence shown in FIG. 3(B). Grey or white circles represent the A or G genotype, respectively. Each horizontal row of squares represents the results from individual alleles. In the sperm, only G alleles were hypermethylated whereas the A alleles are hypomethylated. Mosaicism was evident with 10 of 16 G alleles demonstrating hypomethylation.

FIG. 5 provides the results of analysis of hMLH1 methylation in normal bowel tissue from cancer patients in Example 1. (A) This photograph shows an example of the COBRA screening assay in the normal bowel tissue from 14 cancer patients. Shown are the results from the hMLH1 C region COBRA. In this subset, one patient showed methylation of hMLH1 in the C region in normal bowel tissue, demonstrated by digestion of the PCR product (upper band) to yield two smaller fragments, which appear as one band (arrow). (B) This figure shows the results of bisulfite allelic sequencing of the fragment from the normal bowel shown in (A). Black and white squares represent individual CpGs and are numbered according to their location in the sequence shown in FIG. 3B. White circles represent the G genotype. Each horizontal row of squares represents the results from individual alleles. Hypermethylated alleles were clearly present and were always of the G genotype. This patient is a GG homozygote for the hMLH1 SNP thus mosaicism cannot be determined.

FIG. 6 provides representative bisulfite sequencing of MSP products from healthy individuals assayed in Example 2. Each horizontal row of squares represents the results from individual alleles. Black and white squares represent individual CpGs that are either methylated, or unmethylated, respectively. Hypermethylated alleles were clearly present in healthy individuals in both the hMLH1 (A) and p16%) genes.

### DETAILED DESCRIPTION OF THE INVENTION

The present invention provides an assay for assessing the risk of disease in an individual, wherein said assay comprises the steps of;

(i) isolating a population of cells from normal tissue of said individual, and
(ii) quantitatively determining the frequency of epimutation of a particular gene in said population of cells, wherein the epimutation of said gene is associated with said disease and said gene is other than one that is subject to normal parent of origin-specific expression.

The determined epimutation frequency in the population of cells is predictive of disease risk (i.e. predictive of a predisposition to said disease) in said individual. For example, a positive risk of disease (i.e. a predisposition to disease) may be predicted by a determined epimutation frequency of at least 1 in 1×10⁶ cells or, more preferably, at least 1 in 1×10³ cells or, most preferably, at least 1 in 5×10² cells. Predictive frequencies of the epimutation may vary according to the source of the cells assayed. That is, the cells used in the assay may be from normal tissues such as, for example, normal peripheral blood, normal hair follicles and normal tissue from the buccal cavity, and determined frequencies which are predictive of disease risk may vary across those different normal tissue types.

As used herein, the term “normal tissue” refers to any tissue which is substantially healthy and not showing any significant symptoms or signs of disease (e.g. the tissue is not cancerous) and includes all normal somatic tissues. As indicated above, the cells used in the assay may be from normal peripheral blood, normal hair follicles and normal tissue from the buccal cavity. In addition to these, cells suitable for assaying may be from other normal somatic tissues including normal colonic mucosa. Preferably, the cells used in the assay are from normal peripheral blood.

The assayed epimutation may be any of the well known epigenetic modifications including DNA methylation (or other covalent modification of DNA), histone and chromatin structure changes (e.g. histone methylation, acetylation, phosphorylation or ubiquitination), or association of other proteins in a complex with DNA of the affected locus (e.g. HP1 and homologues).

The assayed epimutation is one which is associated with the disease for which a predisposition is to be assessed. For example, the epimutation is present in a chromosomal locus comprising a gene implicated in the manifestation or development of a disease. Table 1 provides a list of genes implicated in over sixty diseases and the epimutation may therefore be one present in a chromosomal locus comprising at least one of the listed implicated genes, the assay thereby being for the assessment of the disease associated with that gene(s). The assayed epimutation may be present in the promoter of the gene(s) or other regulatory region of the gene(s) and is associated with transcriptional silencing of the gene(s).

Preferably, the assayed epimutation is one which is associated with cancer. More preferably, the assayed epimutation is present in a tumour suppressor gene such as hMLH1, hMSH2, APC 1A, APC 1B and p16.

Most preferably, the assayed epimutation is present in hMLH1.

The determination of the epimutation frequency in the assayed population of cells may be achieved by either directly assaying methylated cytosine (or other modification) on individual DNA strands or by otherwise assaying pooled DNA/chromatin, without examining individual DNA strands.

In order that the nature of the present invention may be more clearly understood, preferred forms thereof will now be described with reference to the following non-limiting example.

### EXAMPLE 2

### Materials and Methods

### Patient Samples

188 individuals with a personal history of cancer from St Vincent's Hospital (Sydney, NSW, Australia) and a further 50 individuals from the Victorian Clinical Genetics Service (Melbourne, VIC, Australia) were included in this study. Of these individuals, 65 were mutation-negative following screening for deleterious germ-line changes in hMSH2, hMLH1 or APC, while 18 had hyperplastic polyposis and the remaining 155 only had a personal history of colorectal cancer.

DNA was extracted from peripheral blood, histologically-normal colonic mucosa, buccal smears, hair follicles and sperm using a standard phenol chloroform procedure (Sambrook et al 1989). To exclude the possibility of contaminating somatic cells in the sperm, semen was sorted on a FACSVantage DiVa (Becton Dickinson, Lexington, Ky., USA) prior to DNA extraction. Sperm were identified on the basis of DNA content after propidium iodide staining as described (Schoell et al 1999). Purity of the sorted sperm was verified by FACS and microscopy.

### MSI Analysis

Prior to the extraction of DNA from paraffin-embedded tissues, an adjacent section was examined histologically to ensure that it contained more than 60% tumour tissue. If this was not the case, foci of tumour were microdissected. The microsatellite status of each tumour was determined as previously described using the following primer sets: Bat 25, Bat 26, Bat 40, D5S346, D2S123, and D17S250 (Ward et al 2001). Tumours with instability at two or more markers were considered microsatellite unstable, while all others were designated as microsatellite stable (MSS).

Immunohistochemical Staining for hMSH2 and hMLH1

Immunohistochemical analysis was performed in a DAKO autostainer on dewaxed 4 μm paraffin sections (DAKO Corporation, Carpinteria, Calif., USA). Staining for hMLH1 and hMSH2 was as previously described, using monoclonal anti-human hMLH1 antibody (1:200, Becton Dickinson, Lexington, Ky., USA) and monoclonal anti-human hMSH2 antibody (1:400, Pharmingen, San Diego, Calif., USA). Expression of hMLH1 or hMSH2 was considered to be absent where there was no staining of tumour cells in the presence of nuclear staining in nearby germinal follicle lymphocytes or in epithelial cells in the base of adjacent non-neoplastic crypts. The immunostaining analysis was reported without knowledge of results of MSI, germ-line or CpG methylation results.

### Methylation Screening Assays

Genomic DNA (2 μg) from each sample was then subject to bisulfite modification (Frommer et al 1992). COBRA (combined bisulfite and restriction analysis; Xiong and Laird 1997) was used as the screening test for all genes. DNA was screened for epimutations in a panel of 10 candidate gene promoters, namely CDKN2A, hMLH1, HPP1, HIC1, RASSF1A, BRCA1, APC 1A and 1B, Blm and O⁶MGMT PCR primers, reaction conditions and post-PCR analysis information for the relevant assays are shown in Table 2. A maximum of 100 ng of bisulfite treated DNA was used in each reaction. In each PCR, positive and negative controls were included, and these were the cell line RKO (gift from M Brattain) and the peripheral blood DNA of a healthy donor, respectively.

Allelotyping of the hMLH1 Promoter

The single nucleotide polymorphism (SNP) at position −33 relative to the hMLH1 transcription start site was determined by RFLP analysis of unmodified genomic DNA. Primers, reaction conditions and restriction digestion details are shown in Table 2.

### Bisulfite Allelic Sequencing

Samples demonstrating a positive result on the COBRA screening assay were examined in more detail using bisulfite sequencing. For hMLH1, this involved a degenerate PCR designed to amplify both unmethylated and methylated alleles, followed by cloning (pGEM-T, Promega) and sequencing of individual alleles with the BigDyes system (ABI). The primers and PCR conditions for amplification of this fragment are shown in Table 2. This fragment was designed to include the G/A SNP described above.

Bisulfite-modified sperm DNA was analysed with a hybrid PCR strategy, using a methylation-specific 5′ primer and a methylation-degenerate 3′ primer, to enrich for methylated sequences (Table 2).

### Results and Discussion

Methylation of hMLH1 is Present in all Adult Tissue from Two Individuals

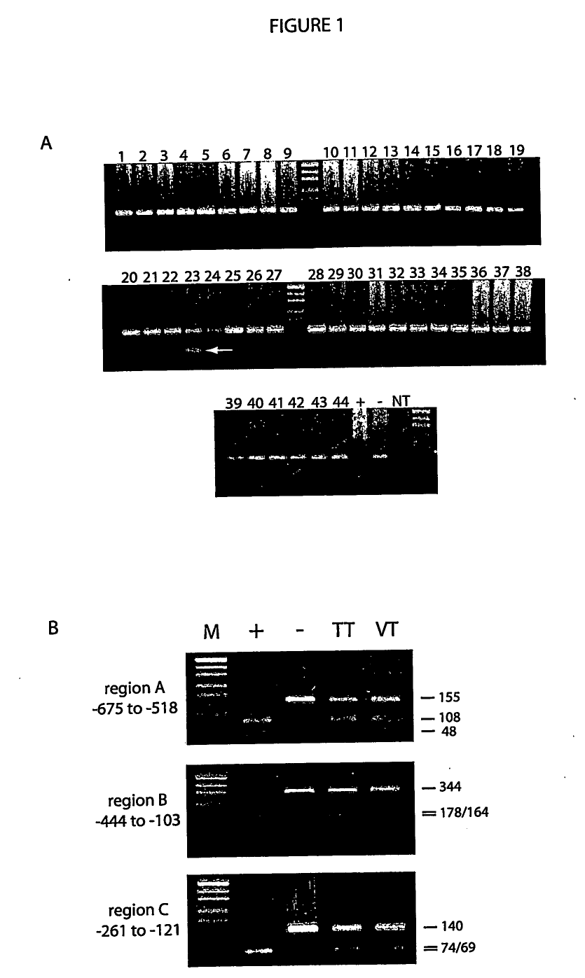

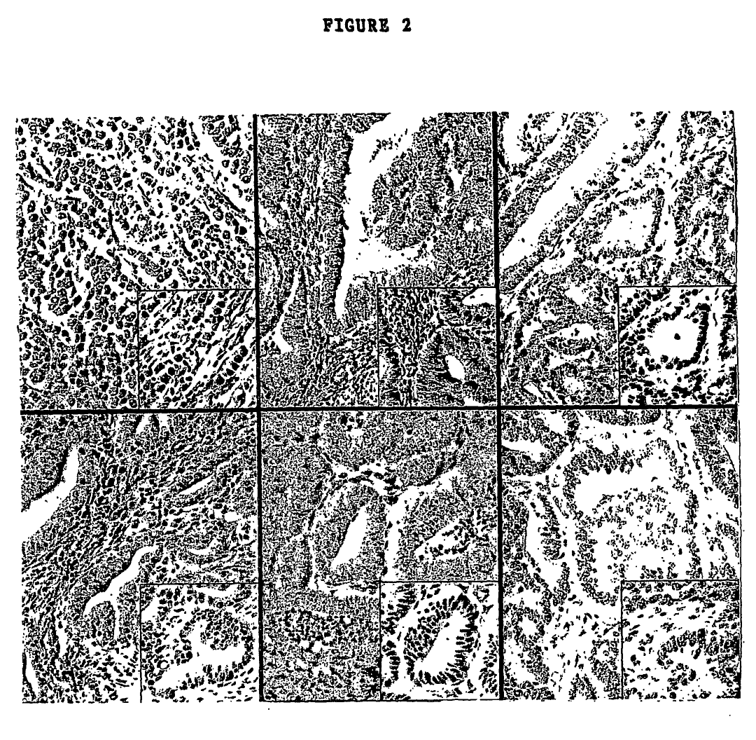

Methylation at hMLH1 was detected in DNA from peripheral blood cells of two of 94 individuals screened (FIG. 1A). The methylation extended across the entire hMLH1 promoter, which is encompassed by regions named A, B and C (FIG. 1B). Similar results were found when the A, B and C screening assays were applied to DNA derived from these patients' hair follicles, and buccal mucosa. These unrelated individuals, TT (male) and VT (female) were aged 64 and 65 years, and both had a personal history of multiple primary malignancies which had been successfully treated with surgery alone. Clinical details of these individuals, as well as the results of immunostaining for the mismatch repair proteins and microsatellite testing of their tumours are shown in Table 3. All tumours tested exhibited microsatellite instability (MSI), which is a hallmark of mismatch repair deficiency. Loss of hMLH1 protein was confirmed in each tumour by immunohistochemistry in the presence of normal staining for hMSH2 (FIG. 2). While TT and VT had previously undergone germ-line testing for mutations in the mismatch repair genes, no deleterious mutations had been identified despite extensive screening with a number of different methodologies.

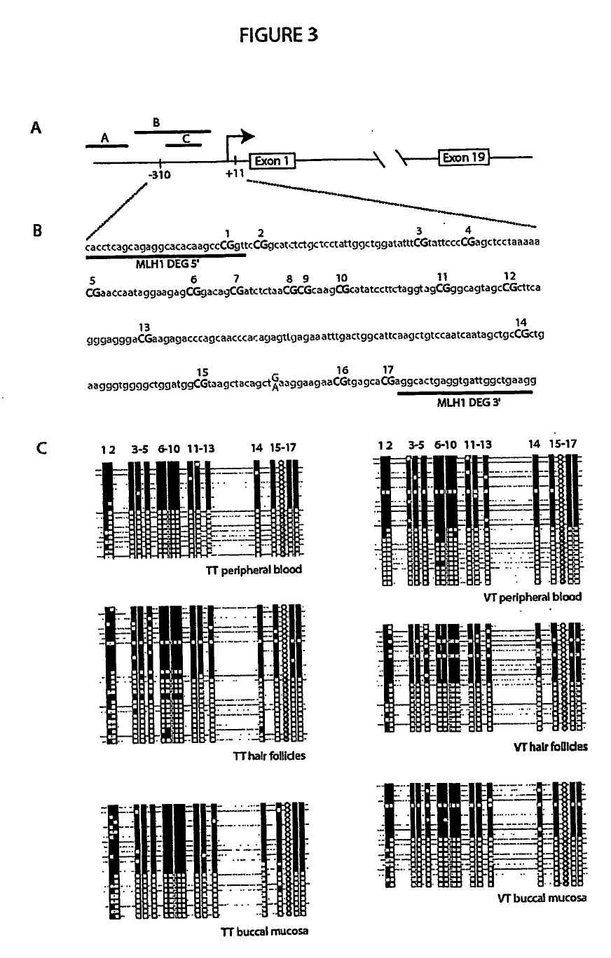

To determine the distribution of CpG methylation within the tissues of TT and VT, and construct detailed methylation maps, bisulfite allelic sequencing was performed. By RFLP analysis, both TT and VT were found to be heterozygous for a G/A single nucleotide polymorphism in hMLH1 at position −33 relative to the transcription start site. Bisulfite sequencing confirmed heterozygosity and also revealed that the methylation in all tissues tested was restricted to the one parental allele (FIG. 3). In both individuals, the affected allele was the G genotype. The A allele was never found to be hypermethylated in any tissue, although some CpG sites were occasionally methylated on some alleles. The significance of this patchy methylation is unknown, but it is not likely to be associated with hMLH1 silencing.

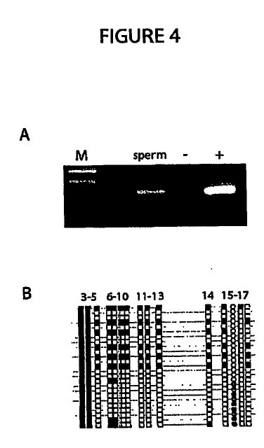

Mosaic methylation was observed in certain tissues from both patients (FIG. 3). Patient VT was found to harbour some hypomethylated G alleles in her peripheral blood (1/15), hair follicles (2/12) and buccal mucosa (1/11). Of somatic tissues, patient TT exhibited mosaicism in hair follicles only, with 1 out of 15 G alleles being hypomethylated. Mosaicism was most evident in the sperm from TT. While the COBRA assay yielded variably weak and negative results (data not shown), methylated alleles were clearly present when the sperm were tested using an MSP-COBRA hybrid PCR (FIG. 4). Of the alleles amplified, only G alleles (6/16) showed hypermethylation. This is not an indication of frequency however, because of the biased nature of the hybrid PCR. However, based on the limit of sensitivity of the assays, it is estimated that between 1 in 500-1 in 1000 alleles may be methylated in the sperm of patient TT.

Methylation of hMLH1 in Colonic Mucosa of Individuals with Cancer

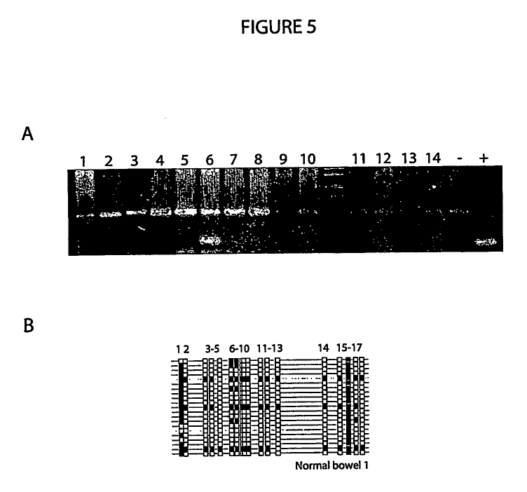

Normal colon tissue (n=133) from individuals with colorectal cancer arising either sporadically or in the setting of hyperplastic polyposis, was also screened. One individual with CpG methylation of hMLH1 in the normal colon tissue was identified (FIG. 5A). This individual (NB1, a 65 yr old male), was assessed in more detail with bisulfite sequencing (FIG. 5B), and was found to be homozygous for the G SNP at −33 in hMLH1, thus rendering the determination of the level of mosaicism impossible. However, sequencing did confirm that NB1 harboured a significant percentage of hypermethylated hMLH1 alleles in his normal colon (i.e. 14%; 3/21 alleles). It was also possible to assess the peripheral blood, hair and buccal mucosa of NB1 for hMLH1 methylation by COBRA, although all assays were negative. Patient NB1 had developed a renal cell cancer at age 57 years and synchronous colorectal cancers in the caecum (microsatellite unstable) and sigmoid colon (microsatellite stable) at the age of 63 years in the setting of hyperplastic polyposis.

### Methylation of Other Genes

All tested patients were negative for methylation at CDKN2A, hMLH2, HPP1, HIC1, RASSF1A, BRCA1, APC 1A and 1B, Blm and O⁶MGMT.

### EXAMPLE 2

### Methods and Materials

### Blood Samples

Peripheral blood was collected from 22 healthy blood donors.

DNA was extracted from the peripheral blood as described in Example 1.

### Methylation Screening Assays

Genomic DNA (2 μg) from each peripheral blood sample was subjected to bisulfite modification as described in Example 1. The bisulfite-treated DNA was then subjected to PCR with methylation-specific primers (i.e. primers that hybridise with DNA in which the CpGs within the primer binding sites are methylated) for the hMLH1 locus and the p16 locus (a tumour suppressor gene). Details of the primers used are listed in Table 4.

### Results and Discussion

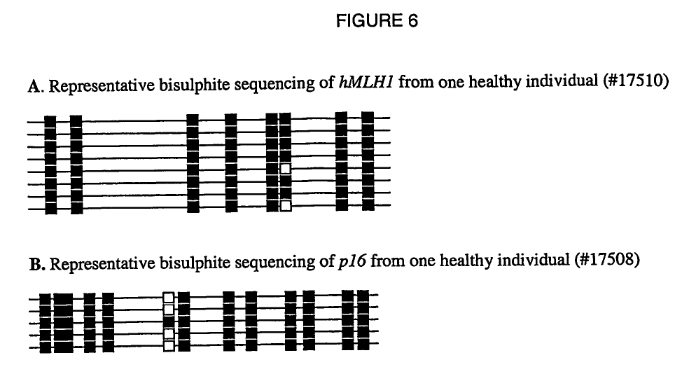

Of the 22 healthy blood donors, 12 had some detectable level of hypermethylated hMLH1 as demonstrated by the presence of a specific product following PCR with methylation-specific primers. For the p16 gene, 18 of 29 healthy blood donors showed a detectable level of hypermethylated alleles. For both genes, the PCR products were confirmed as being hypermethylated by bisulfite sequencing. Representative sequencing is shown in FIG. 6.

The results of this example indicate that healthy individuals commonly carry a detectable level of cells in which the hMLH1 or p16 gene is epimutated. Inactivation of one allele of either hMLH1 or p16, and indeed many other tumour suppressor genes, is known to predispose a cell to become malignant through loss or inactivation of the second allele. In Example 1, two cancer patients (i.e. VT and TT) were described who carry an epimutation in all or nearly all of their somatic cells; the normal individuals studied in this example therefore presumably carry the epimutation in only a small proportion of their somatic cells. It is, however, considered that these cells are at risk of becoming malignant through loss or inactivation of the second allele, and this risk is higher than that of cells that do not carry the epimutation. Thus, it follows that the more cells in an individual that carry the epimutation, the higher will be that individual's risk of developing cancer; which is analogous to disease caused by mosaic carriage of a genetic mutation. Thus, the risk of developing cancer may be assessed by measuring the proportion of somatic cells carrying a particular epimutation. Also, the risk of developing other diseases that result from germline epimutation (particularly, if the disease results when only one allele is inactivated, i.e. haploinsufficiency, or when only a proportion of somatic cells are affected by this loss, i.e. mosaicism) ought similarly be assessed by measuring the proportion of somatic cells carrying a particular epimutation.

TABLE 1
List of human diseases and genes associated with these disease phenotypes

| | | Genes Implicated (reference |
|---|---|---|
| | | from Online Mendelian |
| System Category | Disease | Inheritance in Man - OMIM) |
| Blood Disease | Gaucher Disease | acid-beta glucosidase; |
| | Hemophilia A | HEMA (Factor VIII); |
| | Hemophilia B | Factor IX; Sphingomyelin |
| | NiemannPick Disease | phosphodiesterase-1; |
| | Paroxysmal Nocturnal | PIG-A; |
| | Hemoglobinuria | SLC11A3; TFR2; HLA-H |
| | Haemochromatosis | |
| | von Willebrand disease | |
| Cancers | Including breast and ovarian cancer, | BRCA1; BRCA2; BRCA3; |
| | malignant melanoma, multiple | APC; hMLH1; hMSH2; |
| | endocrine neoplasia, | hMSH6; hPMS1; hPMS2; |
| | neurofibramatosis, pancreatic | CDKN2; MEN1; NF1; NF2; |
| | cancer, polycystic kidney disease, | DPC4; PKD1; HPC1 locus; |
| | prostate cancer, retinoblastoma, | Rb; E cadherin; hamartin; |
| | leukaemia, lymphoma, tuberous | tuberin; VHL; PRKARIA; |
| | sclerosis, von Hippel-Lindau | PTEN; MMAC1; TEP1; |
| | syndrome, gastric cancer, renal | CDK4; MET; SDHB; SDHC; |
| | cancer, endometrial cancer, | SDHD; BMPR1A; p53; RET; |
| | paraganglioma, | PTC; WT; LKB1 |
| | phaeochromocytoma; basal cell | |
| | carcinoma; soft tissue sarcoma; brain | |
| | tumours; testicular cancers; | |
| | gynaecological malignancies; | |
| Digestive System | Ulcerative colitis; Crohn's Disease | CD19; sialophorin; CD11; IL- |
| | Juvenile Onset Diabetes | 4 receptor |
| | Wilson's Disease | IDDM1; IDDM2; IDDM3, -4, -5, |
| | | -6, -7, -10, -12, -13, 15, -18; |
| | | GCK; INSR; AVPR2; ATP7B |
| Eye, Ear, Nose, | Deafness; Pendred Syndrome; Best | connexin 26; pendrin; |
| and Throat | Disease; Glaucoma; Gyrate Atrophy | VMD2; GLC1A |
| | of the Choroid and Retina | ornithine ketoacid |
| | | aminotransferase |
| Endocrine | Adrenoleukodystrophy | ABCD1 |
| | Autoimmune Polyglandular | AIRE |
| | Syndrome | CSA; CSB |
| | Cockayne Syndrome | HNF-4α; glucokinase; HNF- |
| | Type II diabetes | 1α; IPF-1; HNF-1β; |
| | | NUEROD1; IRF-1; IPF1 |
| Cardiovascular | Ataxia Telangiectasia | ATM |
| | Long QT Syndrome | KCNQ1 |
| | Dilated cardiomyopathy | dystrophin, lamin A/C; |
| | Hypertrophic cardiomyopathy | cardiac |
| | Tangier Disease | actin |
| | Essential hypertension | βmyosin heavy chain; α |
| | Eclampsia | tropomyosin; cardiac |
| | Atherosclerosis | troponin T and I; myosin |
| | | binding protein C; myosin |
| | | light chain; calcineurin; |
| | | ABC1; |
| | | angiotensinogen; β2 |
| | | adrenergic receptor; alpha |
| | | adducin; angiotensin- |
| | | converting enzyme; serum |
| | | paraoxonase; endothelial |
| | | nitric oxide synthase; PAI-1; |
| | | fibrinogen B; MTHFR; |
| | | mineralocorticoid receptor |
| | | ApoE; LDL receptor; hepatic |
| | | lipase; |
| Immune System | DiGeorge Syndrome | DGS |
| | Immunodeficiency with Hyper-IgM | TNFSF5 |
| | IgA deficiency | 6p21.3 (locus) |
| | Severe Combined | IL2RG; JAK3; ADA |
| | Immunodeficiency | MHC Class II genes |
| | Autoimmune diseases including | CTLA-4; IL-2; AIRE |
| | SLE, Sjogren syndrome, | |
| | scleroderma, Type I diabetes, coeliac | |
| | disease, inflammatory bowel | |
| | disease, autoimmune haemolytic | |
| | anaemia | |
| Muscle and Bone | Amyotrophic Lateral Sclerosis | SOD1 |
| | CharcotMarieTooth Syndrome | CMT loci |
| | Duchenne Muscular Dystrophy | dystrophin |
| | Marfan Syndrome | FBN1 |
| | Osteoarthritis | |
| Nervous System | Alzheimer Disease | PS1; PS2 |
| | Amyotrophic Lateral Sclerosis | SOD1; CNTF |
| | Angelman Syndrome | UBE3A |
| | Epilepsy | EPM2A |
| | Parkinson Disease | alpha-synuclein; UCHL1 |
| | Phenylketonuria | parkin |
| | Prader-Willi Syndrome | PAH |
| | Refsum Disease | SNRPN |
| | Rett Syndrome | PAHX |
| | Schizophrenia | MeCP2 |
| | Spinal Muscular Atrophy | Neuregulin; reelin |
| | Tay-Sachs Disease | SMN1 |
| | | HEXA |

TABLE 2
Methods for amplification of various hMLH1 loci

| ASSAY | | | | PRODUCT | POST-PCR |
|---|---|---|---|---|---|
| TYPE | LOCUS | PRIMERS AND SEQUENCES 5′-3′ | PCR CYCLING* | SIZE | ANALYSIS |
| COBRA | MLH1-A | MLH1 AF | 1× 94° C. 5 min | 155 bp | Digest product |
| | −691 to | TTAYGGGTAAGTYGTTTTGAYGTAGA | 10× 94° C./30 sec; 65° C./60 sec | | with 2U Tai1. |
| | −442 | (SEQ ID NO: 1) | (−1°/cycle) | | If methylated, |
| | | MLH1 AR | 25× 94° C./30 sec; 55° C./45 sec | | product will |
| | | CCTATACCTAATCTATCRCCRCCTCA | | | digest to |
| | | (SEQ ID NO: 2) | | | yield 108 and |
| | | | | | 48 bp |
| | | | | | fragments |
| | MLH1-B | MLH1 BF | 1× 94° C. 5 min | 344 bp | Digest product |
| | −444 to | AAATTTTTTAATTTTGTGGGTTG | 10× 94° C./30 sec; 60° C./60 sec | | with 2U Hind1. |
| | −103 | (SEQ ID NO: 3) | (−1°/cycle) | | If methylated, |
| | | MLH1 BR | 25× 94° C./30 sec; 50° C./45 sec | | product will |
| | | ACTTCCATCTTACTTCTTTTAAAC | | | digest to |
| | | (SEQ ID NO: 4) | | | yield 178 and |
| | | | | | 164 bp |
| | | | | | fragments |
| | MLH1-C | MLH1 CF | 1× 94° C. 5 min | 140 bp | Digest product |
| | −291 to | GGTTGGATATTTYGTATTTTTYGAG | 10× 94° C./30 sec; 65° C./60 sec | | with 2U BstU1. |
| | −42 | (SEQ ID NO: 5) | (−1°/cycle) | | If methylated, |
| | | MLH1 CR | 25× 94° C./30 sec; 55° C./45 sec | | product will |
| | | AATTACTAAATCTCTTCRTCCCTCC | | | digest to |
| | | (SEQ ID NO: 6) | | | yield 74 and |
| | | | | | 69 bp |
| | | | | | fragments |
| ALLELO- | G/A SNP | MLH1 SNP 5 | 1× 94° C. 5 min | 296 bp | Digest product |
| TYPING | at −33 | GCATCTCTGCTCCTATTGGCTGGATA | 10× 94° C./30 sec; 65° C./60 sec | | with PvuII |
| | | (SEQ ID NO: 7) | (−1°/cycle) | | A/A genotype |
| | | MLH1 SNP 3 | 20× 94° C./30 sec; 55° C./45 sec | | will not |
| | | AGTGCCTTCAGCCAATCACCTCAGTG | | | digest |
| | | (SEQ ID NO: 8) | | | A/G will |
| | | | | | partially |
| | | | | | digest to |
| | | | | | yield 296, 247 |
| | | | | | and 40 bp |
| | | | | | fragments |
| | | | | | G/G will |
| | | | | | digest comp- |
| | | | | | letely to 247 |
| | | | | | and 40 bp |
| ALLELIC | MLH1 | MLH1 DEG 5 | 1× 94° C. 5 min | 324 bp | Product cloned |
| SEQUENCING | −372 to | TATTTTAGTAGAGGTATATAAGTTYGG | 10× 94° C./30 sec; 62° C./60 sec | | into P-GEM-T |
| | −52 | (SEQ ID NO: 9) | (−1°/cycle) | | vector and |
| | | MLH1 DEG 3 | 26× 94° C./30 sec; 52° C./45 sec | | individual |
| | | CCTTCAACCAATCACCTCAATACC | | | clones are |
| | | (SEQ ID NO: 10) | | | sequenced |
| HYBRID | MLH1 | MLH1 MSP 5 | 1× 94° C. 5 min | 276 bp | Product cloned |
| PCR | −326 to | ATTGGTTGGATATTTCGTATTTTTC | 10× 94° C./30 sec; 62° C./60 sec | | into P-GEM-T |
| | −52 | (SEQ ID NO: 11) | (−1°/cycle) | | vector and |
| | | MLH1 DEG 3 | 27× 94° C./30 sec; 52° C./45 sec | | individual |
| | | CCTTCAACCAATCACCTCAATACC | | | clones are |
| | | (SEQ ID NO: 12) | | | sequenced |

*All reactions performed with FastStart Taq polymerase with 1 μM each primer, 2 mM MgCl₂ and 0.25 mM dNTPs

TABLE 3
Results of microsatellite testing and immunostaining
on tumours from two individuals (VT and TT) with
hMLH1 methylation of Example 1.

| | | | MLH1 | MSH2 | |
|---|---|---|---|---|---|
| | Cancer | Age | IHC | IHC | MSI |
| TT | Colorectal - caecum | 43 | Neg | Pos | H |
| | Colorectal - descending colon | 44 | ND | ND | ND |
| | Duodenal - 3 synchronous tumours | 51 | Neg | Pos | H |
| | Ampulla of Vater | 59 | Neg | Pos | H |
| VT | Colorectal - caecum | 46 | Neg | Pos | H |
| | Endometrial | 53 | Neg | Pos | H |
| | Melanoma | 57 | ND | ND | ND |
| | Breast - infiltrating ductal | 63 | Neg | Pos | H |

The age at which each tumour developed is shown. H = microsatellite instability, ND is not done because of insufficient DNA or tumour was unavailable. Immunostaining is shown as present (Pos), absent (Neg) or not done (ND).

TABLE 4
Primers used in methylation-specific PCR in Example 2

| | Primer sequence | | Product |
|---|---|---|---|
| Locus | 5′-3′ | PCR cycling | size |
| hMLH1 | forward: | 1× 94° C. 5 min | 202 |
| −162 to +40 | TAGTAGTCGTTTTAGGGAGG | 10× 94° C./30 sec; 62° C./60 sec | |
| | GAC | (−1°/cycle) | |
| | (SEQ ID NO: 13) | | |
| | reverse: | 30× 94° C./30 sec; 52° C./45 sec | |
| | AAAAAACGTCTAAATACTC | | |
| | AACGAA | | |
| | (SEQ ID NO: 14) | | |
| p16 | forward: | 1× 94° C. 5 min | 96 |
| +256 to +352 | GTTGGTTACGGTCGCGGTTC | 10× 94° C./30 sec; 65° C./60 sec | |
| | (SEQ ID NO: 15) | (−1°/cycle) | |
| | reverse: | 30× 94° C./30 sec; 55° C./45 sec | |
| | CCGACCGTAACTATTCGATA | | |
| | CG | | |
| | (SEQ ID NO: 16) | | |

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Antequera, F. and A. Bird. 1993. CpG islands. Exs 64: 169-85.
2. Baylin, S. B. and J. G. Herman. 2000. DNA hypermethylation in tumorigenesis: epigenetics joins genetics. Trends Genet 16: 168-74.
3. Bird, A. 2002. DNA methylation patterns and epigenetic memory. Genes Dev 16: 6-21.
4. Botstein, D. and N. Risch. 2003. Discovering genotypes underlying human phenotypes: past successes for mendelian disease, future approaches for complex disease. Nat Genet 33: 228-37.
5. Cubas, P., C. Vincent, and E. Coen. 1999. An epigenetic mutation responsible for natural variation in floral symmetry. Nature 401: 157-61.
6. Cui, H., M. Cruz-Correa, F. M. Giardiello, D. F. Hutcheon, D. R. Kafonek, S. Brandenburg, Y. Wu, X. He, N. R. Powe, and A. P. Feinberg. 2003. Loss of IGF2 imprinting: a potential marker of colorectal cancer risk. Science 299: 1753-5.
7. Cui, H., I. L. Horon, R. Ohlsson, S. R. Hamilton, and A. P. Feinberg. 1998. Loss of imprinting in normal tissue of colorectal cancer patients with microsatellite instability. Nat Med 4: 1276-80.
8. Cui, H., P. Onyango, S. Brandenburg, Y. Wu, C. L. Hsieh, and A. P. Feinberg. 2002. Loss of imprinting in colorectal cancer linked to hypomethylation of H19 and IGF2. Cancer Res 62: 6442-6.
9. Duhl, D. M., H. Vrieling, K. A. Miller, G. L. Wolff, and G. S. Barsh. 1994. Neomorphic agouti mutations in obese yellow mice. Nat Genet 8: 59-65.
10. Frommer, M., L. E. McDonald, D. S. Millar, C. M. Collis, F. Watt, G. W. Grigg, P. L. Molloy, and C. L. Paul. 1992. A genomic sequencing protocol that yields a positive display of 5-methylcytosine residues in individual DNA strands. Proc Natl Acad Sci USA 89:1827-31.
11. Herman, J. G., A. Umar, K. Polyak, J. R. Graff, N. Ahuja, J. P. Issa, S. Markowitz, J. K. Willson, S. R. Hamilton, K. W. Kinzler, M. F. Kane, R. D. Kolodner, B. Vogelstein, T. A. Kunkel, and S. B. Baylin. 1998. Incidence and functional consequences of hMLH1 promoter hypermethylation in colorectal carcinoma. Proc Nat Acad Sci USA 95:6870-5.
12. Holliday, R. 1987. The inheritance of epigenetic defects. Science 238: 163-70.
13. Jenuwein, T. and C. D. Allis. 2001. Translating the histone code. Science 293: 1074-80.
14. Jones, P. A. and P. W. Laird. 1999. Cancer epigenetics comes of age. Nat Genet 21: 163-7.
15. Jones, P. A. and D. Takai. 2001. The role of DNA methylation in mammalian epigenetics. Science 293: 1068-70.
16. Kouzarides, T. 2002. Histone methylation in transcriptional control. Curr Opin Genet Dev 12: 198-209.
17. Morgan, H. D., H. G. Sutherland, D. I. Martin, and E. Whitelaw. 1999. Epigenetic inheritance at the agouti locus in the mouse. Nat Genet 23: 314-8.
18. Risch, N J. 2000. Searching for genetic determinants in the new millennium. Nature 405: 847-56.
19. Sambrook, J., E. F. Fritsch, and T. Maniatis. 1989. Molecular cloning. A laboratory manual. Cold Spring Harbor Laboratory Press, Cold Spring Harbor.
20. Schoell, W. M., M. Klintschar, R. Mirhashemi, and B. Pertl. 1999. Separation of sperm and vaginal cells with flow cytometry for DNA typing after sexual assault. Obstet Gynecol 94: 623-7.
21. Tamaru, H. and E. U. Selker. 2001. A histone H3 methyltransferase controls DNA methylation in Neurospora crassa. Nature 414: 277-83.
22. Ward, R., A. Meagher, I. Tomlinson, T. O'Connor, M. Norrie, R. Wu, and N. Hawkins. 2001. Microsatellite instability and the clinicopathological features of sporadic colorectal cancer. Gut 48: 821-9.
23. Wheeler, J. M., N. E. Beck, H. C. Kim, I. P. Tomlinson, N. J. Mortensen, and W. F. Bodmer. 1999. Mechanisms of inactivation of mismatch repair genes in human colorectal cancer cell lines: the predominant role of hMLH1. Proc Natl Acad Sci USA 96: 10296-301.
24. Wolffe, A. P. and M. A. Matzke. 1999. Epigenetics: regulation through repression. Science 286:481-6.
25. Xiong, Z. and P. W. Laird. 1997. COBRA: a sensitive and quantitative DNA methylation assay. Nucleic Acids Res 25: 2532-4.

1. An assay for assessing the risk of disease in an individual, wherein said assay comprises the steps of;
(i) isolating a population of cells from normal tissue of said individual, and
(ii) quantitatively determining the frequency of epimutation of a particular gene in said population of cells, wherein the epimutation of said gene is associated with said disease and said gene is other than one that is subject to normal parent of origin-specific expression.

2. The assay of claim 1, wherein the normal tissue is selected from the group consisting of normal peripheral blood, normal hair follicle tissue and normal tissue from the buccal cavity.

3. The assay of claim 1, wherein the normal tissue is normal peripheral blood.

4. The assay of claim 1, wherein the epimutation is DNA methylation.

5. The assay of claim 1, wherein the epimutation is present in the promoter or other regulatory region of the gene and is associated with transcriptional silencing of said gene.

6. The assay of claim 1, wherein the epimutation is associated with cancer.

7. The assay of claim 1, wherein the epimutation is present in a tumour suppressor gene.

8. The assay of claim 7, wherein the epimutation is present in a gene selected from the group consisting of hMLH1, hMSH2, APC1A, APC1B and p16.

9. The assay of claim 8, wherein the epimutation is present in hMLH1.


SEQUENCE LISTING (17 sequences)

SEQ ID NO: 1 - DNA, 26 bp, Artificial Sequence
  Description of Artificial Sequence Synthetic primer
ttaygggtaagtygttttgaygtaga

SEQ ID NO: 2 - DNA, 26 bp, Artificial Sequence
  Description of Artificial Sequence Synthetic primer
cctatacctaatctatcrccrcctca

SEQ ID NO: 3 - DNA, 23 bp, Artificial Sequence
  Description of Artificial Sequence Synthetic primer
aaattttttaattttgtgggttg

SEQ ID NO: 4 - DNA, 24 bp, Artificial Sequence
  Description of Artificial Sequence Synthetic primer
acttccatcttacttcttttaaac

SEQ ID NO: 5 - DNA, 25 bp, Artificial Sequence
  Description of Artificial Sequence Synthetic primer
ggttggatatttygtatttttygag

SEQ ID NO: 6 - DNA, 25 bp, Artificial Sequence
  Description of Artificial Sequence Synthetic primer
aattactaaatctcttcrtccctcc

SEQ ID NO: 7 - DNA, 26 bp, Artificial Sequence
  Description of Artificial Sequence Synthetic primer
gcatctctgctcctattggctggata

SEQ ID NO: 8 - DNA, 26 bp, Artificial Sequence
  Description of Artificial Sequence Synthetic primer
agtgccttcagccaatcacctcagtg

SEQ ID NO: 9 - DNA, 27 bp, Artificial Sequence
  Description of Artificial Sequence Synthetic primer
tattttagtagaggtatataagttygg

SEQ ID NO: 10 - DNA, 24 bp, Artificial Sequence
  Description of Artificial Sequence Synthetic primer
ccttcaaccaatcacctcaatacc

SEQ ID NO: 11 - DNA, 25 bp, Artificial Sequence
  Description of Artificial Sequence Synthetic primer
attggttggatatttcgtatttttc

SEQ ID NO: 12 - DNA, 24 bp, Artificial Sequence
  Description of Artificial Sequence Synthetic primer
ccttcaaccaatcacctcaatacc

SEQ ID NO: 13 - DNA, 23 bp, Artificial Sequence
  Description of Artificial Sequence Synthetic primer
tagtagtcgttttagggagggac

SEQ ID NO: 14 - DNA, 25 bp, Artificial Sequence
  Description of Artificial Sequence Synthetic primer
aaaaaacgtctaaatactcaacgaa

SEQ ID NO: 15 - DNA, 20 bp, Artificial Sequence
  Description of Artificial Sequence Synthetic primer
gttggttacggtcgcggttc

SEQ ID NO: 16 - DNA, 22 bp, Artificial Sequence
  Description of Artificial Sequence Synthetic primer
ccgaccgtaactattcgatacg

SEQ ID NO: 17 - DNA, 322 bp, Homo sapiens
cacctcagcagaggcacacaagcccggttccggcatctctgctcctattggctggatatttcgtattccccgagctcctaaaaacgaaccaataggaagagcggacagcgatctctaacgcgcaagcgcatatccttctaggtagcgggcagtagccgcttcagggagggacgaagagacccagcaacccacagagttgagaaatttgactggcattcaagctgtccaatcaatagctgccgctgaagggtggggctggatggcgtaagctacagctraaggaagaacgtgagcacgaggcactgaggtgattggctgaagg